(19) United States
(12) Patent Application Publication
(10) Pub. No.: US 2020/0078055 A1
(43) Pub. Date: Mar. 12, 2020

(54) ACCESSORY FOR TENSIONING AN ELONGATE ELEMENT

(71) Applicant: COUSIN BIOTECH, WERVICQ SUD (FR)

(72) Inventors: Guy DENEUVILLERS, MERLIMONT (FR); Jules PRANDI, TOURCOING (FR); Farid KAMCHE, TOURCOING (FR)

(21) Appl. No.: 16/609,858

(22) PCT Filed: May 4, 2018
(86) PCT No.: PCT/EP2018/061478

§ 371 Date: Oct. 31, 2019

(30) Foreign Application Priority Data
May 4, 2017 (FR) 1753949

(51) Int. Cl. A61B 17/70

(52) U.S. Cl. CPC A61B 17/7074; A61B 2017/00407; A61B 17/7053

(57) ABSTRACT

The present invention concerns an accessory for tensioning an elongate element for attaching an implant to a bone element, comprising a rod having proximal and distal ends and a longitudinal axis L, the distal end of said rod being provided with a bearing device for bearing on the implant. The accessory further comprises a device for tensioning the elongate element, comprising a drum mounted so as to be able to rotate about an axis T, transverse to the axis L, arranged at the proximal end of the rod, the drum comprising a holding portion suitable for cooperating with the elongate element. The tensioning device comprises a device for rotating the drum about the axis T, allowing the elongate element to be tensioned by winding the elongate element around the drum.

### BACKGROUND OF THE INVENTION

The present invention relates to the technical field of ancillaries for tensioning an elongate, in particular a flat, element for the attachment of an implant on a bone element, in particular at least one portion of a vertebral body.

It is known to correct the abnormal curvatures of the spine and/or the pathological inclinations of the vertebrae relative to each other by the use of implantable longitudinal bars disposed on either side of the vertebral column and secured to the vertebrae by means of screws inserted into the vertebrae themselves or of hooks, introduced for example along the spinal canal. These screws and hooks are however aggressive for the spine.

To overcome these drawbacks, it has been proposed a flexible elongate attachment element held in tension by means of an implant, which implant is optionally configured to be secured to a longitudinal bar, the elongate element attached to the implant being engaged with at least one vertebral portion by forming a loop therearound.

The implantable longitudinal bars can be implemented in the treatment of scoliosis or the treatment of degenerative pathologies.

Different levels of degeneration are found, for example on a vertebral level such as a discopathy or on two vertebral levels such as the spondylolisthesis or even on more than three levels such as the C or S scoliosis.

In these treatments, the longitudinal bar can be put in connection with the vertebral level to be corrected by means of an elongate element, which extends around at least one portion of a vertebral body and around said bar then the ends of said elongate element can be held by means of a suture, a knot or using an implantable securing means. Said securing means may comprise a housing receiving the longitudinal bar and the elongate element in the wound state therearound, and clamping means for blocking the bar and said elongate element in said housing. Generally, the clamping means comprise means for pinching said elongate element thereby preventing any movement of the latter. It is also possible not to use an implantable longitudinal bar but only an elongate element extending around at least one portion of a vertebral body, and in particular on one or more vertebral level(s), said elongate element being held in tension by means of an implant securing its two ends together.

One of the problems that the present invention seeks to solve is to tension the elongate element when it forms a loop on at least one portion of a vertebral body, and is engaged with an implant and/or an implantable longitudinal bar.

Document EP 1.933.743 131 relates to an ancillary for tensioning an elongate element comprising a movable cylindrical part able to move in translation around a rod, said rod comprising, at its distal end, bearing means on a longitudinal bar. The movable cylindrical part also comprises a lug for the attachment of the elongate element. The dynamometric clamping system is operable by means of a handle. The tensioning of the elongate element is performed by the surgeon by pressing the handle without limitation in the tension that can be applied. The surgeon can thus apply too much tension on the elongate element, which may affect the adjustment of the longitudinal bar(s) placed on the vertebral level(s) to be corrected, or even move vertebrae. In addition, the tension is applied on a loop in the extension of the two free ends of the elongate element that are secured by means of a keeper. The tension applied will therefore not be the same on each of the free ends since it is applied directly on a single free end of the elongate element. The application of the tension is therefore unbalanced on each free end of the elongate element and may cause erosion of the vertebral portion on which the tension is applied and/or slipping of the elongate element on said vertebral portion.

Document FR 2.981.841 A1 relates to a tensioning ancillary trying to prevent, compared to document EP 1.933.743 131, the elongate element from twisting when it is tensioned. The ancillary thus comprises a return so as the elongate element forms an angle of 90°. The ends of the elongate element provided with securing means are attached to a compensation wheel according to two diametrically opposite points. The tension applied to each of the ends of the elongate element is therefore not balanced. In addition, the return at a 90° angle of the elongate element as well as the means for attaching an end in a groove generate significant frictions on the elongate element which may weaken it. In addition, because of the return member, the ancillary is cumbersome. However, during a surgical procedure, multiple holding implants and longitudinal bars are placed on the vertebral level(s) to be corrected, so that it is important for the surgeon, once an elongate element is tensioned, to have easy access to the implant for holding the elongate element in order to complete the clamping and the attachment thereof on the elongate element but also to be able to access other holding implants and/or implantable longitudinal bars.

Document WO 2016/051088 relates to a tensioning ancillary comprising a movable carriage mounted on a rotary shaft disposed about the longitudinal axis of the body of the ancillary, the rotating of the shaft driving in translation the carriage in connection with the elongate element.

In the aforementioned documents, the tensioning is achieved by displacement of a movable element, in connection with the elongate element, in translation along a longitudinal axis. These tensioning ancillaries comprise complex and voluminous tensioning devices in particular because of the space requirement of the clamping stroke according to which the movable element translates.

### OBJECT AND SUMMARY OF THE INVENTION

The present invention thus relates to a tensioning ancillary:
for tensioning an elongate element forming a loop on at least one portion of a vertebral body without damaging said vertebral body, which is gradual and easily adjustable by the surgeon;
for exerting an equal tension on each of the two ends of the elongate element;
allowing the surgeon to quickly release the tension exerted on the elongate element if necessary;
which is compact, thus facilitating its use, and the possible disposition of several tensioning ancillaries on the same operation site;
of simplified construction so as it is for single use only.

The present invention thus relates, according to a first aspect, to an ancillary for tensioning an elongate element for the attachment of an implant on a bone element, in particular at least one portion of a vertebral body, by surrounding at least partially said bone element, said ancillary comprising a rod having proximal and distal ends and a longitudinal axis L, the distal end of said rod being provided with a bearing device to bear on the implant, said ancillary further comprises a device for tensioning said elongate element. Advantageously, the tensioning device comprises a drum rotatably mounted about an axis T, transverse to the axis L, disposed at the proximal end of the rod and a holding portion configured to hold the elongate element. The tensioning device further comprises a device for driving in rotation the drum about the axis T, which makes it possible to tension the elongate element by winding of the elongate element around the drum.

Advantageously, the elongate element first adopts a rectilinear path along the longitudinal axis L of the rod and is then tensioned by winding thereof around the drum. The tensioning device is therefore very compact, which reduces its space requirement on the operation site. This disposition is also advantageous for its storage.

The tensioning of the elongate element is achieved by winding of the latter around the drum which is driven in rotation by the driving device, the tensioning is therefore gradual.

Advantageously, the periphery of the drum forms an area for storing the elongate element in the wound state as it is tensioned. This disposition considerably limits the space requirement of the tensioning device compared to the tensioning ancillaries of the state of the art for which the tensioning is achieved by displacement in translation of the movable element, in connection with the elongate element, along an axis.

This disposition also makes it possible to have more freedom in the choice of the length of the elongate element since the clamping stroke, formed around the drum, is less bulky than the clamping strokes in the state of the art.

Advantageously, the arrangement of the tensioning device according to the proximal end of the rod facilitates its manipulation and allows moving it away from the operation area.

It is understood in the present text by “the axis T is transverse to the axis L” that these two axes intersect, particularly the axis T is substantially perpendicular to the axis L.

Preferably, the distance separating the distal and proximal ends of the rod is determined so that the tensioning device is not, under use conditions, in contact with the site of the surgical procedure. Only the distal end, by means of the bearing device, and optionally a portion of the rod in the extension of its distal end, are likely to come into contact with the site of the procedure when the bearing device bears on an implant.

Preferably, the distance separating the distal and proximal ends of the rod is greater than or equal to 5 cm, more preferably greater than or equal to 10 cm.

Preferably, the bearing device is configured to bear on an implant, whether the latter is an implantable longitudinal bar or an implant for holding in tension the elongate element, said implant being optionally configured to be also secured to a longitudinal bar.

In operation, the elongate element is placed in connection with the holding portion for it to be held to the drum, then the device for driving in rotation the drum is actuated, particularly manually, so as to drive in rotation the drum and to achieve the tensioning of the elongate element by winding. When the driving device is no longer actuated, the rotating and therefore the tensioning are stopped.

The implant for holding in tension the elongate element in the wound state on at least one portion of a vertebral body may be the one described in the international patent application PCT/FR2014/051801 filed in the name of the Applicant, and whose description is incorporated in the present text, or have a different configuration, in particular not comprising means for securing to an implantable longitudinal bar.

Preferably, the bearing device is molded to form a housing receiving, by interlocking, an implant for holding in tension of the elongate element in the wound state on at least one portion of a vertebral body.

Preferably, the bearing device and/or the rod and/or at least partly the tensioning device and/or the drum, and/or at least partly the rotation driving device, are in a polymeric, in particular reinforced, in particular molded, material.

The construction of the ancillary according to the invention being simplified, it is thus possible to manufacture the ancillary by molding large part of the parts constituting it.

Particularly, the polymeric material comprises one or more polymers, in particular in admixture with reinforcing elements.

The polymer(s) may be selected from: polyethylene terephthalate, polycarbonate, polyarylamide, polyamide (such as polyamide 4-6, polyamide 6-6, polyamide 6, polyamide 12 or 11).

The reinforcing elements are preferably reinforcing fibers, mote preferably made of glass or carbon, in particular glass.

The carbon fibers comprise in particular a mass proportion of carbon greater than or equal to 90%.

The polymeric material may thus be a polyarylamide filled with fiberglass, such as the one marketed under the trademark IXEF® by Solvay.

Preferably, the ancillary is for single use. This disposition is facilitated because of the molding of all or part of the parts forming the ancillary.

This disposition limits the risks of spread of the germs, and therefore of infection for the patients, particularly by nosocomial diseases.

Indeed, the tensioning ancillaries in the state of the art are sterilized for reuse, but this sterilization not only represents an extra cost but it proves to be difficult given their construction involving many parts.

Advantageously, the tensioning ancillary according to the invention, when it is for single use only, avoids a sterilization operation for reuse, thus limiting the risks of infection.

The portion for holding the drum is configured to hold one or two free end(s) of the elongate element.

Preferably, the elongate element is a textile element, which may be braided, knitted or woven, preferably braided. Still preferably, the elongate element is flat in order to generate significant frictional forces with the surfaces against which it is moving, thus improving the clamping forces. This disposition also limits the abrasion of the surfaces in contact with the elongate element in comparison with a thin and substantially cylindrical elongate element.

The invention is proposed thereafter in a series of variants, which can be considered alone or in combination with one or several of the preceding ones.

In one variant, the rotation driving device comprises a lever pivotally mounted about the axis T, and a clutch device configured so that the pivoting of the lever along the direction of rotation R1 in a first position drives in rotation the drum about the axis T.

This disposition facilitates the gripping of the driving device, and improves its ergonomics. Preferably, the lever is a handle having a shape converging towards its front portion.

In one variant, the driving device comprises a clutch-release device configured so that, in the declutched position, the lever being pivoted about the axis T along a direction of rotation R2 opposite to the direction of rotation R1, the drum is freely rotatable about the axis T.

This disposition makes it possible to release the tension exerted on the elongate element.

This operation can be carried out at the end of clamping to be able to cut the elongate element as close as possible to the implant for holding in tension the elongate element in the wound state on at least one portion of a vertebral body, disposed in operation at the bearing device.

In one variant, the driving device comprises a return device configured so that, once the lever is released from the pressure exerted thereon for it to pivot along the direction of rotation R1 in the first position, the lever pivots along a direction of rotation R2, opposite to the direction of rotation R1, in a rest position.

This disposition allows performing a stepwise clamping. The actuation of the lever winds by a determined length the elongate element around the drum, thereby allowing its clamping. It is therefore necessary to actuate the lever several times until the desired tensioning is achieved.

In one variant, the driving device comprises a non-return device configured to prevent rotation of the drum along a direction of rotation R2, opposite to the direction of rotation R1, in the first position and optionally in the rest position.

In one variant, the driving device comprises a gripping clamp comprising first and second portions, the first portion forming said lever and the second portion being fixed with the rod.

Preferably, the first portion and said second portion each comprise proximal and distal portions, said proximal portions each having a convergent shape along a direction opposite to their distal portions, in particular in the form of a handle.

Preferably, the second portion, having proximal and distal portions, is in the extension of the rod, still preferably the bearing device for the bearing on the implant and/or the rod and/or the second portion is/are molded in one piece, particularly in a polymeric (reinforced or not) material as defined above.

Preferably, the distal portion of the second portion has a casing shape (also described hereinafter) configured to receive in rotation the drum.

In one variant, the approximation of the first and second portions of the gripping clamp, by pivoting of the first portion forming the lever towards the second portion, causes the rotation of the drum.

This disposition further improves the ergonomics of the ancillary, by facilitating the manual actuation of the driving device.

In one variant, the clutch device is a ratchet mechanism, particularly comprising a first toothed wheel, and optionally a second toothed wheel, in connection, rotatably about the axis T, with the drum and a ratchet part in connection with the lever.

Particularly, the ratchet part is in movable connection with the lever.

Advantageously, the ratchet part is configured to cooperate with the first toothed wheel, and optionally with the second toothed wheel, so that the pivoting of the lever along the direction of rotation R1 in a first position meshes the ratchet part, in connection with the lever, with the first toothed wheel, and optionally the second toothed wheel, thereby rotating the toothed wheel(s) and the drum.

Preferably, the connection between the ratchet part and the lever is configured so that the ratchet part has a high position in which the ratchet part is disengaged from the toothed wheel(s), and a low position in which the ratchet part cooperates with the teeth of the toothed wheel(s).

Particularly, the ratchet part in connection with the lever is in a low position when the lever is pivoted along the direction of rotation R1 in the first position, and optionally when the lever is in the rest position.

Particularly, the ratchet part is in a high position, when the lever is in the declutched position.

Particularly, the ratchet part in connection with the lever alternates the high and low positions, on the one hand, during its passage from the first position to the rest position, and during its passage from the rest position to the declutched position.

Preferably, the ratchet part is in connection with the lever by means of a stud attached to said ratchet part and slidably mounted in a slide attached to the lever, further preferably a spring is disposed around the stud.

In one variant, the non-return device comprises a ratchet part, in particular in connection with the second portion of the clamp, configured to cooperate with the first toothed wheel, and optionally the second toothed wheel.

Preferably, the connection between the ratchet part and the second portion is configured so that the ratchet part has a high position, in which the ratchet part is disengaged from the toothed wheel(s), and a low position, in which the ratchet part cooperates with the toothed wheel(s).

Particularly, the ratchet part in connection with the second portion is in a low position when the lever is in the first position, and optionally in the rest position.

Particularly, the ratchet part in connection with the second portion is in a high position, when the lever is in the declutched position.

Particularly, the ratchet part, in connection with the second portion, alternates the high and low positions, during pivoting of the lever along the direction of rotation R1 in the first position.

Preferably, the ratchet part is in connection with the second portion by means of a stud attached to said ratchet part and slidably mounted in a slide attached to the second portion, preferably a spring is disposed around the stud.

In one variant, the tensioning device comprises a casing receiving the drum, said casing being in fixed connection with the proximal end of the rod (particularly said casing forms the distal portion of the second portion of the clamp).

In one variant, the drum comprises two lateral ends, and the casing comprises right and left cheeks each having an opening receiving a lateral end of the drum.

In one variant, the lever comprises a proximal portion (configured to be grippable) and a distal portion, said distal portion comprises two lateral projections between which the casing is disposed, and optionally the first and second toothed wheels disposed on either side the casing.

In one variant, the clutch-release device comprises at least one cam, preferably two cams, particularly supported by the casing, configured to deactivate the ratchet mechanism, particularly to disengage the ratchet part in connection with the lever from the first toothed wheel, and optionally from the second toothed wheel.

In one variant, the clutch-release device comprises at least one cam, preferably at least two cams, particularly supported by the lever, more particularly by the distal portion of the first portion forming the lever, configured to disengage the ratchet part in connection with the second portion from the first toothed wheel, and optionally from the second toothed wheel.

The two aforementioned variants, considered alone or in combination, make it possible to place the lever in the declutched position.

In one variant, the lever (in particular its distal portion) and/or the casing each comprise two through slots each receiving a lateral end of a ratchet part, with which it is/they are in connection. Said slots are preferably configured so that the lateral ends of the ratchet part(s) translate therein, in particular from a high position to a low position.

In one variant, the holding portion is a through slot of the drum allowing the passage of at least one free end, preferably both free ends, of the elongate element.

Advantageously, it is not necessary to secure the elongate element by the use of complex and tedious securing means to implement. The surgeon thus places at least one free end of the elongate element through the slot of the drum, and then the winding of the elongate element on itself during the rotating of the drum allows the blocking of the elongate element on itself and its securing to the drum.

The present invention relates, according to a second aspect, to an assembly comprising a tensioning ancillary according to any one of the preceding variants according to a first aspect, and at least one implant, particularly chosen from an implantable longitudinal bar or an implant for holding in tension the elongate element in the wound state on at least one portion of a vertebral body.

The present invention will be better understood upon reading an exemplary embodiment described below, cited in a non-limiting manner and illustrated by the drawings described hereinafter appended to the present text.

### DETAILED DESCRIPTION OF THE DRAWINGS

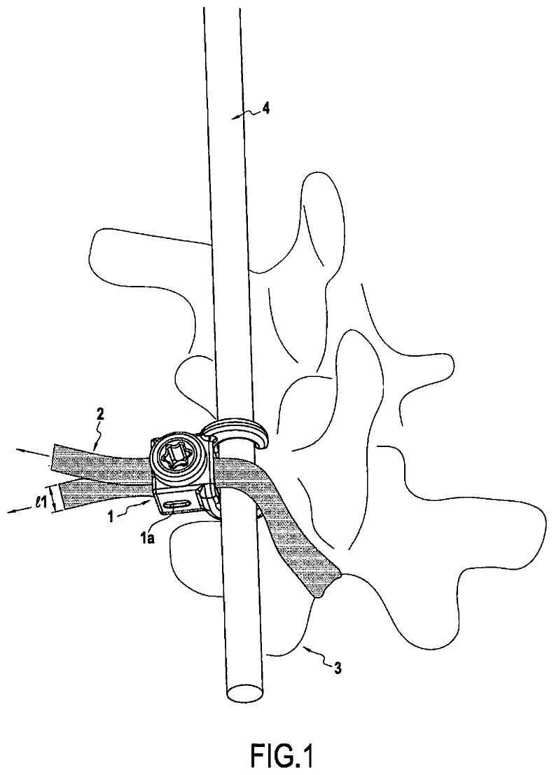

FIG. 1 is a schematic representation in perspective of an example of an implant for holding in tension the elongate element in the wound state on at least one portion of a vertebral body also comprising means for securing to a implantable longitudinal bar;

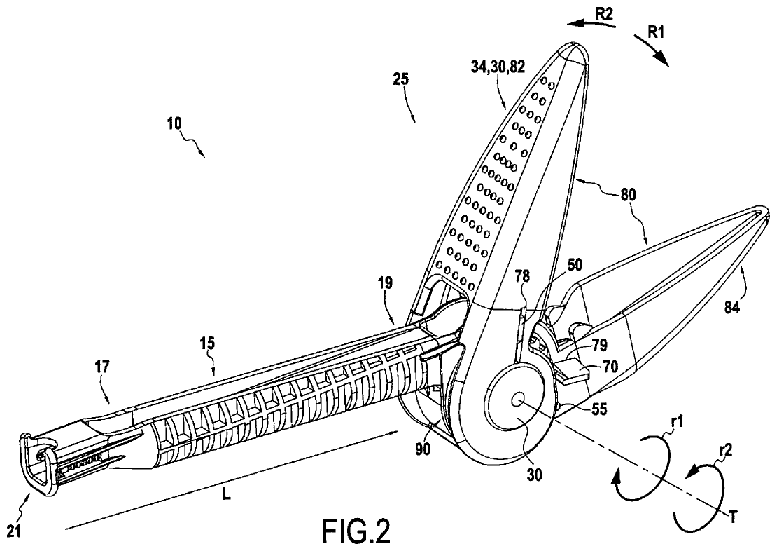

FIG. 2 is a schematic representation in perspective of an example of a tensioning ancillary according to the invention;

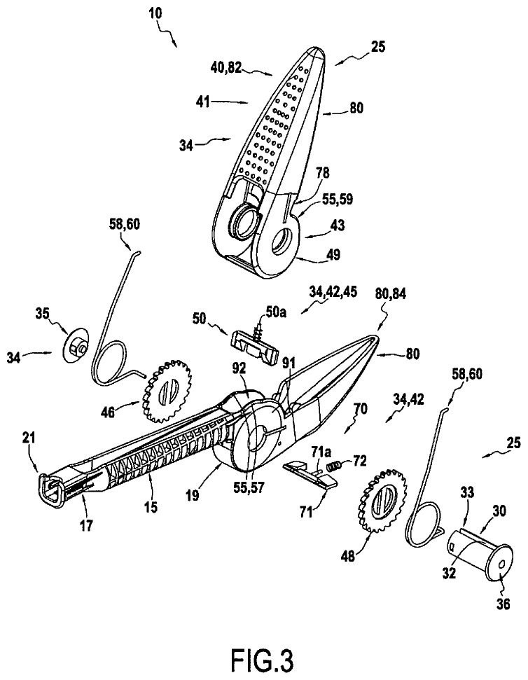

FIG. 3 is a schematic representation in perspective of an exploded view of the tensioning ancillary represented in FIG. 2;

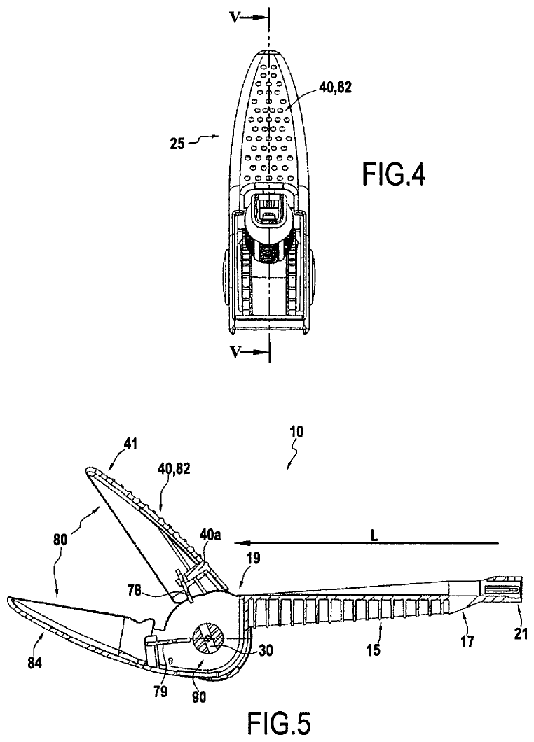

FIG. 4 is a schematic representation, viewed from above, of the lever of the tensioning device according to the invention of the ancillary represented in FIGS. 2 and 3;

FIG. 5 is a schematic representation of the ancillary whose tensioning device is represented according to the sectional plane V-V in FIG. 4;

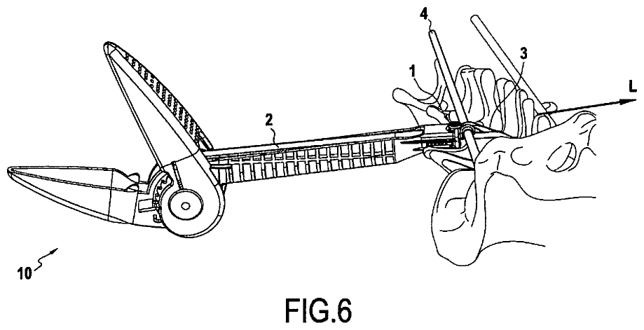

FIG. 6 is a schematic representation in perspective of an assembly according to the present invention comprising the tensioning ancillary represented in FIGS. 2 to 5, in operation, and the implant for holding in tension of the elongate element in the wound state on at least one portion of a vertebral body also comprising means for securing to an implantable longitudinal bar.

### DETAILED DESCRIPTION OF THE EXAMPLES OF EMBODIMENT

FIG. 1 represents an implant for holding in tension 1 the elongate element 2 in the wound state on at least one portion of a vertebral body 3, said implant 1 comprising, in this specific example, also means for securing 5, 6 to an implantable longitudinal bar 4. Said flat elongate element 2 having a width 11, prior to its attachment in its wound state around the portion of the vertebral body 3 has been tensioned using a tensioning ancillary according to the invention.

The example of a tensioning ancillary 10 according to the invention, represented in FIGS. 2 to 6, comprises a rod 15 having distal 17 and proximal 19 ends and a longitudinal axis L, the distal end 17 of said rod 15 being provided with a bearing device 21 to bear on the implant 1. The ancillary 10 further comprises a device for tensioning 25 said elongate element 2. The tensioning device 25 comprises a drum 30 rotatably mounted about the axis T, transverse to the axis L, particularly substantially perpendicular thereto, disposed at the proximal end 19 of the rod 15 and comprises a holding portion 32 adapted to cooperate with the elongate element 2 for the latter to be held. The tensioning device 25 further comprises a device for driving in rotation 34 the drum 30 about the axis T, which makes it possible to tension the elongate element 2 by winding of the elongate element 2 around the drum 30.

The driving device 34 comprises a lever 40 pivotally mounted about the axis T, and a clutch device 42 configured so that the pivoting of the lever 40 along the direction of rotation R1 in a first position drives in rotation the drum 30 about the axis T.

The clutch device 42 is a ratchet mechanism 45 comprising a first toothed wheel 46 and a second toothed wheel 48, each being in connection, rotatably about the axis T, with the drum 30, as well as a ratchet part 50 in connection, particularly movable connection, with the lever 40. The ratchet part 50 is in connection with the lever 40 by means of a stud 51 attached to said ratchet part 50 and slidably mounted in a slide 400 attached to the lever 40, still preferably a spring 52 is disposed around the stud 51.

The driving device 34 also comprises a clutch-release device 55 configured so that, in the declutched position, the lever 40 being pivoted about the axis T along a direction of rotation R2 opposite to the direction of rotation R1, the drum 30 is freely rotatable about the T axis.

The driving device 34 comprises a return device 58 configured so that, once the lever 40 is released from the pressure exerted thereon for it to pivot along the direction of rotation R1 in the first position, the lever 40 pivots along a direction of rotation R2, opposite to the direction of rotation R1, in a rest position.

The driving device 34 comprises a non-return device 70 configured to prevent rotation of the drum 30 along a direction of rotation R2, opposite to the direction of rotation R1, when the lever 40 is in the first position, and in the rest position.

The driving device 34 also comprises a gripping clamp 80 comprising first 82 and second 84 portions, the first portion 82 being said lever 40 and the second portion 84 being fixed with the rod 15.

The second portion 84 comprises a proximal portion 85 and a distal portion 86. The distal portion 86 forms the casing 90. The proximal portions 85 and 41, respectively of the first 82 and second 84 portions, are convergent and form each a gripping handle.

The non-return device 70 comprises a ratchet part 71, in particular in connection with the second portion 84 of the clamp 80, configured to cooperate with the first toothed wheel 46, and the second toothed wheel 48. The ratchet part 71 is in connection, particularly movable connection, with the second portion 84 by means of a stud 72 attached to said ratchet part 71 and slidably mounted in a slide (not represented) attached to the second portion 84, preferably a spring 73 is disposed around the stud 72.

The tensioning device 25 comprises a casing 90 receiving the drum 30, said casing 90 being in fixed connection with the proximal end 19 of the rod 15.

The lever 40 comprises a proximal portion 41 and a distal portion 43, said distal portion 43 comprises two lateral projections 44, 49 between which the casing 90 is disposed, and the first 46 and second 48 toothed wheels being disposed on either side of the casing 90.

The drum 30 comprises two lateral ends 31, 33, and the casing 90 comprises right 91 and left 92 cheeks each having an opening, only the opening 93 is visible in FIG. 3, receiving a lateral end 31, 33 of the drum 30.

The drum 30 also comprises two lateral closure caps 35, 36 securing in rotation the drum 30 to the lever 40 and to the second portion 84.

The clutch-release device 55 comprises two cams 57 mounted on the casing 90, particularly outer cam profiles 57, configured to deactivate the ratchet mechanism 45, particularly to disengage the ratchet part 50 in connection with the lever 40 from the first toothed wheel 46, and from the second toothed wheel 48.

The clutch-release device 55 further comprises two cams 59 mounted on the lever 40, particularly outer cam profiles 59, more particularly mounted on the distal part 43 of the first portion 82 forming the lever 40, configured to disengage the ratchet piece 71, in connection with the second portion 84, from the first toothed wheel 46 and from the second toothed wheel 48.

The clutch-release device 55 also comprises two pairs of slots 78, 79 receiving the lateral ends of the ratchet parts 50 and 70.

In one variant, the holding portion 32 is a through slot of the drum 30 allowing the passage of at least one free end, preferably both free ends, of the elongate element 2.

In this specific example, the return device 58 comprises two return springs 60 rotatably mounted about the axis T. A portion of each of the springs is mounted on the drum 30 and another longitudinal portion of each of the springs 60 is configured to bear on and exert a restoring force on the lever 40 along the direction of rotation R2.

In operation, at least one end of the elongate element 2, preferably the two free ends, coming from the tension holding implant 1, is/are passed in the holding portion 32, particularly through the through slot, then the user approaches the first 82 and second 84 portions to the gripping clamp 80, by pivoting of the first portion 82 forming the lever 40 toward the second portion 84. This pivoting causes the rotation of the drum 30, and therefore the winding of the elongate element 2 on itself, which self-blocks and is therefore held at the holding portion 32. The first portion 82 of the clamp 80 is pivoted until it is in contact with the second portion 84, in the first position. During the pivoting of the first portion 82 corresponding to the lever 40, the lever 40 switches from its rest position, in which the ratchet part 50 and the ratchet part 71 are in the low position, that is to say mesh the first 46 and second 48 toothed wheels, to the first position, in which the ratchet part 50 remains in the low position and the ratchet part 71 switches from the low position to the high position as a function of the advance of the toothed wheels 46 and 48 thereby ensuring the anti-return disposition function 70. When the user releases the lever 40, the latter under the effect of the return device 58, returns to its rest position, the ratchet part 50 then adopts high and low positions while the ratchet part 71 remains in the low position thus preserving the winding of the elongate element 2 performed around the drum 30. The user pivots the lever 40 of the clamp 80 towards its second portion 84, switching from the rest position to the first position, as many times as necessary until obtaining the desired winding of the elongate element 2 around the drum 30 and thus the appropriate tensioning of the elongate element 2 around said at least one portion of the vertebral body. The user then locks the tension holding implant 1 in order to hold in the clamped state the elongate element 2 on at least one longitudinal bar and at least one vertebral body portion.

The user then pivots the lever 40 along the direction of rotation R2, opposite to the direction of rotation R1, until it comes into abutment against the rod 15, in a declutched position. The clutch-release device 55, in particular by means of the set of cams 57 and 59, disengages the ratchet parts 50 and 71 from the toothed wheels 46 and 48 so that the drum 30 is freely rotatable. The user can then cut the elongate element 2 at the implant 1 in order to eliminate the length of elongate element 2 which not necessary to the clamping.

Preferably, all parts of the tensioning anchor 10 are in a molded polymeric material, particularly reinforced using reinforcing fibers. The tensioning ancillary is thus preferably is for single use only, so that once the tensioning is performed, the tensioning ancillary 10 is discarded. It is also possible to recycle the polymeric material.

In this specific example, the bearing device 21 comprises a housing configured to receive the implant 1 by clipping. The bearing device according to the invention is not limited to bear on this type of implant 1, and may be configured in order to cooperate to bear on other types of implants for holding in tension 1 the elongate element 2 in the wound state on at least one portion of a vertebral body, and/or to directly bear on an implantable longitudinal bar.

Advantageously, the bearing device according to the invention can be molded according to the configuration of the implant for holding in tension the selected elongate element.

1-15. (canceled)

16. An ancillary for tensioning an elongate element for the attachment of an implant on a bone element, by surrounding at least partially said bone element, said ancillary comprising a rod having proximal and distal ends and a longitudinal axis L, the distal end of said rod being provided with a bearing device to bear on the implant, said ancillary further comprises a device for tensioning said elongate element, wherein said tensioning device comprises a drum rotatably mounted about an axis T, transverse to the axis L, disposed at the proximal end of the rod and comprising a holding portion adapted to cooperate with the elongate element, and wherein said tensioning device further comprises a device for driving in rotation the drum about the axis T, which makes it possible to tension the elongate element by winding of the elongate element around the drum.

17. the ancillary tensioning according to claim 16, wherein the driving device comprises a lever pivotally mounted about the axis T, and a clutch device configured so that the pivoting of the lever along the direction of rotation R1 in a first position drives in rotation the drum about the axis T.

18. The tensioning ancillary according to claim 17, wherein the driving device comprises a clutch-release device configured so that, in the declutched position, the lever being pivoted about of the axis T along a direction of rotation R2 opposite to the direction of rotation R1, the drum is freely rotatable about the axis T.

19. The tensioning ancillary according to claim 17, wherein the driving device comprises a return device configured so that, once the lever is released from the pressure exerted thereon for it to pivot along the direction of rotation R1 in the first position, the lever pivots along a direction of rotation R2, opposite to the direction of rotation R1 in a rest position.

20. The tensioning ancillary according to claim 17, wherein the driving device comprises a non-return device configured to prevent rotation of the drum along a direction of rotation R2, opposite to the direction of rotation R1, when the lever is in the first position, and optionally in the rest position.

21. The tensioning ancillary according to claim 16, wherein the driving device comprises a non-return device configured to prevent rotation of the drum along a direction of rotation R2, opposite to the direction of rotation R1, when the lever is in the first position, and in the rest position.

22. The tensioning ancillary according to claim 17, wherein the driving device comprises a gripping clamp comprising first and second portions, the first portion being said lever and the second portion being fixed with the rod.

23. The tensioning ancillary according to claim 22, wherein the approximation of the first and second portions of the gripping clamp, by pivoting of the first portion forming the lever towards the second portion, causes the rotation of the drum.

24. The tensioning ancillary according to claim 17, wherein the clutch device is a ratchet mechanism, in connection, rotatably about the axis T, with the drum and a ratchet part in connection with the lever.

25. The tensioning ancillary according to claim 24, wherein the ratchet mechanism comprises a first toothed wheel and a second toothed wheel.

26. The tensioning ancillary according to claim 24, wherein the non-return device comprises a ratchet part, configured to cooperate with the first toothed wheel.

27. The tensioning ancillary according to claim 26, wherein the ratchet part is in connection with the second portion of the clamp, configured to cooperate with the first toothed wheel, and the second toothed wheel.

28. The tensioning ancillary according to claim 16, wherein the tensioning device comprises a casing receiving the drum, said casing being in fixed connection with the proximal end of the rod.

29. The tensioning ancillary according to claim 28, wherein the drum comprises two lateral ends , and wherein said casing comprises right and left cheeks each having an opening receiving a lateral end of the drum.

30. The tensioning ancillary according to claim 29, wherein the lever comprises a proximal portion and a distal portion, said distal portion comprises two lateral projections between which the casing is disposed.

31. The tensioning ancillary according to claim 30, wherein the first and second toothed wheels are disposed on either side of the casing and between the two lateral projections of said distal portion.

32. The tensioning ancillary according to claim 18, wherein the clutch-release device comprises at least one cam, configured to deactivate the ratchet mechanism

33. the tensioning ancillary according to claim 32, wherein the at least one cam is mounted on the casing and is configured to disengage the ratchet part in connection with the lever from the first toothed wheel, and from the second toothed wheel.

34. the tensioning ancillary according to claim 16, wherein the holding portion is a through slot of the drum allowing the passage of at least one free end of the elongate element.

35. An assembly comprising a tensioning ancillary according to claim 16, and at least one implant chosen from an implantable longitudinal bar or an implant for holding in tension the elongate element in the wound state on at least one portion of a vertebral body.
